(19) United States
(12) Patent Application Publication
(10) Pub. No.: US 2011/0212858 A1
(43) Pub. Date: Sep. 1, 2011

(54) SYNTHETIC POLY D/L LYSINE FOR CONTROL OF DIRECTION AND RATE OF NEURITE GROWTH AND REGENERATION

(75) Inventors: Stephen L. Chen, El Monte, CA (US); Richard B. Simerly, Glendale, CA (US)

(73) Assignee: CHILDRENS HOSPITAL LOS ANGELES, Los Angeles (CA)

(21) Appl. No.: 13/127,915

(22) PCT Filed: Nov. 13, 2009
(86) PCT No.: PCT/US2009/064437

§ 371 Date: May 5, 2011

Related U.S. Application Data
(60) Provisional application No. 61/114,800, filed on Nov. 14, 2008.

(51) Int. Cl. C40B 30/06; C40B 60/12; C12N 5/0793

(52) U.S. Cl. 506/10; 506/39; 435/395

(57) ABSTRACT

The present invention provides for use of poly-D/L-lysine (PLL) to control the growth of neural cells in vitro and in vivo. The invention describes the activity of defined PLL lines on neural cells and the ability to use the compound to control the direction and rate of growth of neurites on solid substrates. High-throughput screening assays are provided as are medical devices and therapies for treatment of neuronal injury or malfunction.

### STATEMENT OF GOVERNMENT INTEREST

This invention was made partially with U.S. Government support from the United States National Institutes of Health under Contract No. NS37952 and Contract No. DK65900. The U.S. Government has certain rights in the invention.

### BACKGROUND OF THE INVENTION

1. Field of the Invention

The present invention relates to the field of medicine, and in particular to the field of neurology. More specifically, the invention relates to devices, research tools, and chemical and biological materials for use in medical and scientific research and medical therapy.

2. Description of Related Art

The nervous system controls all bodily functions including muscle movements, bodily secretions and higher mental functions like language and memory. The neuron is the basic cellular unit of the nervous system. The human brain contains billions of neurons that form functional connections through long thin processes on one end of the cell body, called axons, and dendrites on the other end. Together, early stage non-matured axons and dendrites are referred to in the field as “neurites”. Both axons and dendrites are referred to herein as neurites. During development, neurites traverse a variety of chemical environments to find appropriate target cells and form large assemblies of connected neurons that collectively form a neural circuit. Nervous system function is encoded in the pattern of connections and the activity of the neural circuits, which control and coordinate bodily function and behavior.

When an axon of a neuron is cut or interrupted, such as in spinal cord injury or limb amputation, the damaged neuron retains the potential to re-grow the axon (called axon regeneration). But, unfortunately, most regenerated axons fail to reach their targets because they fail to properly navigate to their appropriate targets due to interference of scar tissue. Axon guidance is a process that describes how axons find their way towards targets (e.g., other neurons or muscle cells) and is currently an area of intense investigation. Yet the molecular events in axon regeneration remain largely unknown. Progress in our understanding of axon guidance impacts both basic research on brain development and clinical therapeutic opportunities.

Poly-D/L-lysine (PLL) is a highly positively charged amino acid found naturally in the body, which is involved in tissue elasticity and structure. It has been used widely in medical research as a coating material for microscope slides and plastic dishes used to study the nervous system. Among its appealing properties are its long shelf life at room temperature, low cost, and its unrestricted availability due to high volume chemical synthesis. Furthermore, PLL is currently being used to coat plastics.

Investigations into axon biochemistry and function have been described in the patent and scientific literature. For example, U.S. Pat. No. 6,428,965 to Ginty discloses an isolated protein called Semaphorin which displays axon guidance activity. In addition, U.S. Pat. No. 6,589,257 to Shimizu discloses use of laminin, collagen, and gelatin to coat an artificial tube for supporting nerve regeneration. However, neither of these patents recognize the usefulness of PLL in neurite guidance during growth.

U.S. Pat. No. 5,908,783 to Brewer discloses the use of a copolymer of sequentially alternating lysine and alanine residues to coat glass and plastic for promoting neuronal survival and axon growth. However, Brewer is silent with regard to the possible use of either amino acid alone for neuronal survival or axon growth. Furthermore, currently there is no commercial product using Brewer's patented technology.

Banker et al. (Neurochem Research, 2003 28(11) pp 1639-48), describes a two-step strategy for micro patterning of proteins on a PLL coating to control neurite growth in culture. According to Banker et al., first, a protein called Protein A is printed onto a PLL coated surface. Second, the Protein A substrate is immersed in a solution of a chimeric protein (this chimeric protein is specially prepared via molecular techniques). The process disclosed by Banker et al. is relatively complicated, and is silent with respect the possible use of PLL alone to control neurite growth.

Sang Beom Jun et al. (Journal of Neuroscience Methods, 2006, V160, Issue 2, Page 317-326) provides an advancement over the other publications discussed above. Sang Beom Jun et al. takes advantage of the fact that neuron attachment and growth are dramatically superior on PLL coated areas over naked surfaces. In the Sang Beom Jun et al. strategy, axons are grown on a glass surface. The majority of the glass surface is naked (i.e., not covered) and only a small portion is coated with PLL by micro-printing to provide a PLL partial coating. In the Sang Beom Jun et al. system, the majority of neurons exposed to the treated glass surface do not survive due to the lack of PLL, which has now been found to be required for adherence. While the disclosed use of micro printing for directional control is interesting, Sang Boem Jun et al. fails to appreciate that differential coating by PLL is a more effective way to control neurite growth direction.

Sasoglu, in his thesis issued to Drexel University Library on September 2008, repeats Sang Beom Jun's strategy of PLL micro printing. The Sasoglu approach leads to the death of 50% of the neurons (page 140), now understood to be due to the naked areas on the substrate. In the reported work, a glass surface having a full PLL coating was used as a negative control to show loss of directional growth (page 140). However, Sasoglu fails to recognize that a differentially coated PLL surface can control axon growth direction. Furthermore, Sasoglu fails to recognize the branching inhibitory effect of PLL. Indeed, there is no discussion at all of neurite branching in this document.

Soussou et al. (IEEE Transactions on biomedical engineering, Vol. 54 No. 7 Page 1309) investigated the effect of PLL full coating on neuron branching. However, Soussou et al. does not disclose or suggest the fact that PLL has a potent effect to block neurite branching. In addition, Romanova et al. (FASEB J. 2004 August; 18(11):1267-9. Epub 2004 Jun. 18) compared the effect of PLL uniform coating to PLL pattern coating (which included naked areas) on the branching of neurites. The researchers concluded that, in contrast to uniform substrates, the shape and size of the growth permissive region on a micropatterned substrate play a dominant role in the production of primary neurites and determines their branching pattern in the direction of extension. However, Romanova et al. does not disclose or suggest that PLL differential coating has the capacity to inhibit neurite branching.

Although a large amount of research has been performed to study growth of neurites on coated surfaces, including surfaces coated with PLL, as yet it has not been disclosed or suggested that differential coating, including coating in which at least fifty percent of the surface is coated with PLL, can be successfully used to control the direction of neurite growth. Furthermore, the usefulness of differential coating with PLL in increasing neurite growth rates has not been disclosed or suggested. In prior art printed pattern models, substantial naked areas are present on the treated growth substrate, resulting in death of neurons on the naked areas and growth only on areas of the surface coated with PLL. PLL coating has thus only been shown to be useful in supporting neuron growth. However, the use of PLL as a true guidance signal for directional growth of neurites has not been disclosed or suggested.

### SUMMARY OF THE INVENTION

The present inventors have realized that there exists a need for new compositions and methods for controlled growth of neurites in vitro and in vivo. Accordingly, the present invention provides for the first application of synthetic PLL as a guidance structure to control the direction of neurite growth using controlled differential deposition of PLL on substrates. The invention also provides for the first application of synthetic PLL to enhance the growth rate of neurites using controlled differential deposition of PLL on substrates. Taken from another viewpoint, the invention can be considered as providing for use of synthetic PLL as an inhibitor to block axon branching during axon regeneration. Likewise, from one viewpoint, the invention provides for use of synthetic PLL as an inhibitor to prevent unwanted growth of cut axons, such as in accidental or surgical amputation.

The present invention has numerous uses in clinical and research environments, both in vitro and in vivo. For example, the technology can be provided as a platform for neuron assays for scientific and pharmaceutical research, such as a variety of microchips and microarrays. Furthermore, three-dimensional arrays of neurons, having controlled and characterized growth patterns, can be provided for use in artificial intelligence and biocomputer applications. Alternatively or in addition, the technology can be provided in the form of a medical supply for wounded nerve treatments, such as to guide nerve regeneration. In such embodiments, it can be provided in the form of a biocompatible film, tubing, or cap. For example, stents or other medical devices can be fabricated that allow for controlled neurite growth, allowing proper neuron reconnections with other neurons or muscle cells through otherwise impermeable tissues (e.g., scar tissue).

In a first general aspect, the invention provides a method of controlling growth of neurites. Broadly speaking, the method includes growing neurons on a solid substrate on which PLL is differentially deposited in pre-defined geometries. According to the method, differential deposition of PLL on the solid substrate provides a spatial guidance matrix for controlled directional growth of the seeded neurons, and in particular neurites of the neurons. According to the invention, neurites grow substantially along the ridge of the PLL line deposited on the solid substrate, thus allowing for pre-determined growth patterns for the neurons. The width of the peak of the PLL line of this invention is comparable to the diameter of a neurite or a growth cone (e.g., on the order of 0.5 μm to 1.5 μm). In situations where the PLL is deposited as a single line along the solid substrate, growth of neurites generally follows the PLL deposition line, although some branching may occur as well. However, branching is substantially reduced, as compared to currently available technologies for growing neurons on solid substrates coated with PLL.

According to the present invention, neurite growth in a pre-defined direction represents at least 50% of the growth of neurites, more preferably at least 75%, 90%, 95%, or 99% of the growth of neurites. In certain embodiments, neurite branching is not detectable. In preferred embodiments, two or more PLL deposition lines are deposited on the solid substrate. Growth of the neurites along the path defined by the deposition lines allows for controlled directional growth of neurites. In embodiments, growth of the neurites along the ridge of the PLL line allows for controlled directional growth of neurites.

According to the present invention, a variety of distances between two neighboring PLL lines are possible, without the requirement of reserving naked gaps in between the lines, as is required in prior techniques. That is, the area of raised PLL surface features can reach more than 50% of the surface area of a substrate, more preferably 75%, 90%, or 99% or more of the substrate. The remaining portion of the surface area of the substrate between the PLL lines can be uniformly covered by PLL as well during the process of differential PLL deposition. During the process of neuron growth on the solid substrates of the invention, after being seeded, a neuron regenerates neurites randomly and the neurites grow slowly at beginning if the neuron happens to be in a uniformly coated PLL area. But the slow growth of the random neurites of the neuron becomes faster and directional when they reach a PLL ridge. From experiment data, it appears that, upon contact with a PLL ridge, neurites climb onto the PLL ridge. The random directional growth then ceases, and the neurites grow only along the direction of the PLL ridge.

It is to be understood that the term “line” as used herein is not limited to a straight line between two defined points. Rather, the term is to be understood broadly to indicate any geometrical shape desired, including, but not limited to straight lines, curves (of any type and complexity), circles, polygons, etc. Any geometrical shape that is desired and can be formed using differential deposition of PLL is encompassed by the term “line”. The term “line” is thus used as a general term and is used for the sake of convenience and brevity only. Unless a specific geometric shape is discussed, the term is to be understood in its broadest sense.

In another general aspect, the invention is directed to methods for controlling the rate of growth of neurites. It has been surprisingly found that the rate of neurite growth on solid substrates having differential PLL deposition is increased, as compared to neurite growth on uniformly covered solid substrates or solid substrates containing substantial “naked” areas (i.e., areas not covered by a substance suitable for neuron attachment and growth). In preferred embodiments, the method of controlling growth of neurites is a method of controlling both the directional growth and rate of growth or neurons, primarily through growth of neurites on solid substrates having PLL differentially deposited to form guidance structures for growth.

The invention also provides methods of making a solid substrate for controlling growth of neurons, in particular through growth of neurites. In general, the methods include differentially depositing PLL on a surface of a solid substrate in pre-defined geometries to create guides, ridges, or tracks for directional growth of the neurons. In preferred embodiments, the method also includes coating a portion or the entire surface with a substance suitable for neuron attachment and growth.

As should be evident, the invention provides solid substrates having differentially deposited PLL lines disposed on at least one surface. The solid substrates of the invention can be used in numerous research settings and in therapeutic treatments. For example, solid substrates having differential PLL deposition can be used to investigate the molecular mechanisms involved in neuron growth and interaction. They thus can be used to investigate electrochemical and biochemical communication between neurons and between neurons and other cells, such as muscle cells. Solid substrates having neurons attached to a surface can also be used in therapeutic processes, such as to reform a neural connection that has been broken, such as by injury or due to the aging process.

### BRIEF DESCRIPTION OF THE DRAWINGS

The accompanying drawings, which are incorporated in and constitute a part of this specification, illustrate embodiments and features of the invention, and together with the written description, serve to explain certain principles of the invention.

FIG. 1 is a schematic representation of differential deposition of PLL on a solid substrate to form multiple PLL lines for controlled growth of neurons.

FIG. 2 depicts a system for uniform coating of a solid substrate with PLL.

FIG. 3 depicts a system for differential deposition of PLL on a solid substrate. Panel A depicts deposition of a first PLL surface feature on the solid substrate. Panel B depicts deposition of a final PLL surface feature on the solid substrate. The panels show that sequential deposition of multiple PLL surface features can provide a single solid substrate with multiple parallel raised surface features or lines. Panel C shows schematically controlled directional growth of a neuron along a single line of the solid substrate of Panel B.

FIG. 4 shows photographs depicting neuron growth on solid substrates. FIG. 4a shows the pattern of growth of neurons on a solid substrate uniformly coated with PLL. FIGS. 4b and 4c show patterns of growth of neurons on a solid substrate having differential deposition of PLL in lines on the surface of the solid substrate.

FIG. 5 shows photographs of neurons grown on a solid substrate uniformly coated with PLL (left panel) and neurons grown on a solid substrate differentially coated with PLL to form raised PLL surface features of a defined geometry (curved line), and further having at least 50% of the remaining surface coated with PLL.

FIG. 6 shows a photograph of neurons grown using standard culturing conditions, showing neurite “point contacts” at random positions about the solid substrate.

FIG. 7 shows a photograph of neurons grown on a solid substrate according to the present invention, showing “line contacts” between neurons along defined PLL lines.

FIG. 8 shows the “line contacts” of FIG. 7 in schematic form.

FIG. 9 shows a photograph of “point contacts” between one neurite and three other neurites for neurons grown using standard culturing conditions.

FIG. 10 shows a photograph of “line contacts” between neurons grown along PLL lines according to the present invention.

FIG. 11 shows a schematic of a High Throughput Screening (HTS) assay device according to an embodiment of the invention.

### DETAILED DESCRIPTION OF VARIOUS EMBODIMENTS OF THE INVENTION

Reference will now be made in detail to exemplary embodiments of the invention, data for which is illustrated in some of the accompanying drawings. These exemplary embodiments are intended to be purely exemplary of the invention, and should not be considered as limiting the invention in any way.

Before certain embodiments of the present invention are described in detail, it is to be understood that the terminology used herein is for the purpose of describing particular embodiments only, and is not intended to be limiting. Further, where a range of values is provided, it is to be understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a surface” includes a plurality of such surfaces and reference to “a sample” includes reference to one or more samples and equivalents thereof known to those skilled in the art, and so forth. Furthermore, the use of terms that can be described using equivalent terms include the use of those equivalent terms. Thus, for example, the use of the term “patient” is to be understood to include the terms “subject”, “animal”, “human”, and other terms used in the art to indicate one who is subject to a medical or clinical treatment.

It is known in the art that PLL coating promotes neuron attachment and growth on coverslips. Numerous patents, scientific publications, and commercial products report the use of plastics and glasses fully coated with PLL to culture neurons. Growth on these substrates is generally disordered and random. To provide more controlled growth, researchers have used techniques to deposit PLL on solid substrates in ordered patterns. However, all previous attempts to control directional growth of neurites have been limited to the use of printed micro patterns of a compound on a solid substrate. The techniques that have been used in the past require the presence on the treated surface of non-coated areas to limit neuron growth. Furthermore, where multiple lines are printed according to those techniques, there is a substantial gap between two adjacent lines. The gaps are generally as wide as the width of the printed lines, and can even be as much as twice as wide as the width of the printed lines. These techniques thus provide solid substrates having at most about 50% of the surface area differentially covered with PLL, and more likely at most about 33% of the surface area. Furthermore, the printable width of the PLL lines are usually greater than 10 μm, which has now been found not to be suitable for neurite directional growth control. In addition, the physical transfer of PLL from a stamp or printer head to a substrate surface creates coarse PLL lines that contain imperfections. Practical applications of such approaches are limited due to the high loss of viable cells that do not adhere to the non-coated areas, the large area of uncoated surface, and the unsuitability of the PLL lines to support directional neurite growth.

The present disclosure teaches, for the first time, that differentially coated PLL surfaces can provide neurite directional growth control on solid substrates without the use of non-coated or naked areas on the substrate as a mechanism for controlling neurite growth directionality. Further, the present disclosure teaches that raised PLL surface features can be placed exceptionally close to each other while still providing neurite growth direction signalling. For example, whereas current technologies result in substantial gaps between deposited lines, the present technology can provide solid substrates in which the gaps between deposited lines is less than 50% of the width of the lines.

As such, the present invention provides for solid substrates having less than 50% of the surface non-coated or naked. Where the surface to be coated is a naked surface, PLL can be deposited in multiple raised surface features that cover 50% or more of the surface. Alternatively, the surface can be first coated with a substance that allows for and/or promotes neuron attachment and growth, such as PLL, then the surface is further treated to provide one or more raised PLL surface features. In embodiments where PLL is used to both uniformly coat and create raised surface features on the surface, the combined PLL coating can cover at least 50% of the surface. Yet again, where a neuron attachment and growth promoting substance other than PLL is used to uniformly coat the surface, raised PLL surface features can be disposed on the coated surface such that the PLL surface coating covers at least 50% of the surface area of the surface.

In some embodiments, one or more raised surface features of PLL are disposed on the surface of a solid substrate to cover less than 50% of the surface, and a portion of the remaining surface is uniformly coated with PLL or another substance to provide a solid substrate having at least 50% of the surface area covered by PLL. In other embodiments, at least 50% of the surface is covered by differentially deposited PLL raised surface features and no other portion of the surface is covered. All combinations of surface area coverage by raised PLL surface features, with or without uniform coating with PLL or another neuron attachment and growth promoter are encompassed by the invention.

The invention creates, for the first time, PLL lines at the molecular level. In exemplary embodiments, individual PLL molecules move and deposit themselves without direct physical contact from the operator. The surface features of PLL lines at the molecular level enables PLL line ridges to be made thinner than possible using prior techniques, providing ridges that are in a size ranges compatible with the diameters of neurites and growth cones. The importance of size compatibility has not be recognized or made available using PLL printing in prior techniques.

The present invention addresses a major area of investigation in neurobiology that impacts the understanding of neurological development. It has wide applicability in all areas of neurobiology, including, for example developmental disabilities and mental illness both in children (e.g., autism) and in adults (e.g., depression). Leaders in the field are recognizing that the causes for developmental disabilities and mental illness are likely rooted in neuronal circuit formation. The human brain contains billions of neurons that form neural connections in the body. During development, neurites migrate through varied chemical environments to find appropriate targets (e.g., other neurons or muscle cells). The assemblies of interconnected neurons form a neural circuit. Nervous system functions are encoded in the neural circuits, which control and coordinate bodily functions. Abnormal development of these circuits can lead to abnormally early loss of mental function and mental illness, even if this is only expressed later in life. How neurons influence each other during the development process and what determines how they develop remains poorly understood. The present invention addresses long-felt needs in the art by providing methods and products for controlled growth of neurons for study and therapeutic use.

Prior to the present invention, there were no versatile, controlled, sensitive, and reproducible ways to investigate the various parameters of interest when studying circuit formation. Neurons grow in an irregular fashion in vitro and it takes a skilled operator to measure and follow changes in neuron behavior. The inability to reproducibly grow and study neurons in a controlled manner has long hampered researchers in the field, and has delayed medical applications involving neuron replacement. The present invention addresses these shortcomings and provides new, powerful ways of producing and using neurons and neural circuits and networks.

The invention includes methods for controlling the growth of neurons on a solid substrate. As used herein, the term solid substrate means any solid article of manufacture onto which PLL can be deposited and on which neurons can adhere and grow. A solid substrate thus may be any of a number of natural or man-made products, including, but not limited to, plastics or other polymeric materials (e.g., nylon, polysaccharides, nitrocellulose), glass, and metals. Co-polymers, alloys, and other combinations of substances are encompassed by the term. Solid substrates may be rigid or flexible, and may have one or more surfaces onto which PLL can be deposited and onto which neurons can be attached and grown. Likewise, the size and shape of the solid substrate can vary depending on the particular application of the technology. The practitioner is free to choose any particular solid substrate, size, and shape based on general parameters known in the art and based on considerations that are relevant for the intended application of the present technology.

According to embodiments of the methods of the invention, an appropriate solid substrate is seeded with one or more neurons under conditions that allow for neuron attachment to a surface of the solid substrate. The seeded cells are then permitted to grow on the surface. As used herein, cell growth is any detectable change in cell size or volume. Typically, growth is measured by detecting lengthening of neurites of the cells. Many techniques for detecting growth can be used, and the practitioner is free to select any appropriate technique. Of the techniques available, the simplest to use is optical detection of the length of neurons. In exemplary embodiments, growth is determined by optical inspection of neurons under a microscope after staining or labeling with an appropriate substance, such as a fluorescent or luminescent compound. In the method, growth can be supported by exposing the seeded cells to an aqueous environment that contains nutrients (e.g., carbon and energy sources), gases, and other substances necessary for cell survival and growth.

According to the invention, growth of neurons on the solid substrate is performed in a controlled manner. In particular, growth is controlled in the context of the directionality of extension of neurites, whereby controlled growth results from differential deposition of PLL on the surface of the solid substrate to create raised surface features. Growth of neurites along the ridge of a PLL line formed by the raised surface feature results in neurons having defined, controlled growth characteristics. As such, differential deposition of PLL to create raised surface features provides a novel way to culture neurons in an orderly fashion, whereby neurites grow along pre-determined lines. It is to be understood that the term “direction” is not to be interpreted as meaning “unidirection”. Thus, for example, for a solid substrate having PLL lines deposited linearly across the upper surface, from left to right, neurons growing in the same “direction” are those that grow along the lines from left to right or from right to left.

Directionally controlled growth occurs on solid substrates that are made according to methods that form one or more raised surface features on the solid substrates, where the raised surface features have pre-defined geometric shapes. Preferably, the raised surface features are created through a process referred to herein as differential deposition. The term differential deposition is used to describe a process by which PLL is deposited on a surface of a solid substrate in a non-uniform manner, resulting in one or more raised surface features of PLL on the treated surface. Although various procedures for forming such raised surface features are contemplated by the invention and can be devised by those of ordinary skill in the art, an exemplary method is detailed below.

The invention encompasses solid substrates having one or more surfaces on which raised PLL surface features are present. The raised surface features can be present on an untreated or “naked” surface, or can be present on a surface that is partially of fully coated with a substance that promotes neuron attachment and/or growth. In some exemplary embodiments, at least a portion of the surface of the solid substrate for growth of neurons is coated with a substance that is suitable for neuron attachment and growth at areas of the surface not differentially coated with PLL. Thus, gaps and open spaces between and/or outside of raised PLL surface features can be the uncoated surface of the solid substrate (e.g., glass, plastic) or can be coated, such as by PLL. Preferably, the substance that is used to coat areas of the surface not differentially coated with PLL also controls the directional growth of the neurons, such as PLL. In some embodiments, at least a portion of the solid substrate is uniformly coated (i.e., coated with substantially the same thickness of the substance) with a substance that is suitable for neuron attachment and growth. For example, the portion of the solid substrate coated with the substance can be 20% of the surface area, 30% of the surface area, 40% of the surface area, 50% of the surface area, 60% of the surface area, 70% of the surface area, 80% of the surface area, 90% of the surface area, or 99% or more (e.g., 100%) of the surface area. While numerous substances are suitable, in exemplary embodiments PLL is used to coat the solid substrate. Preferably, PLL is also used to form raised surface features.

Coating can be performed at any time prior to seeding of neurons onto the solid substrate. In exemplary embodiments coating of at least a portion of the solid substrate is performed prior to differential deposition of the PLL raised surface features (i.e., a two-step process for creation of a solid substrate). In other exemplary embodiments, coating of at least a portion of the solid substrate is performed at the time of differential deposition of PLL onto the surface of the solid substrate (i.e., a one-step process for creation of a solid substrate). In highly preferred embodiments, coating of at least a portion of the solid substrate with PLL is used for cell adhesion to the surface of the solid substrate and for cell growth in vitro, while PLL differential deposition is used for controlled directional growth of the neurons. Of course, the raised surface features comprising or consisting of PLL contribute to cell attachment and growth as well.

In addition to at least partial coating of the solid substrate surface, the surface of the solid substrate is treated to develop raised surface features (also referred to herein as “lines”), typically comprising or consisting of PLL. The process of differential deposition is used to form these surface features on the solid substrate in pre-defined geometries. The surface features extend above the plane of the solid substrate and represent a directional signal for growth of neurites. More specifically, a property of the PLL lines that the present invention exploits is the growth cone signaling conferred by the peak or ridge of the PLL line. While the molecular mechanism of action of PLL in this regard is not fully understood, it is thought that the three-dimensional shape of the PLL ridge provides a signal to the neurons to grow along the PLL ridge. It can be postulated that the neurons detect the ridge of the PLL line and preferentially grow along that ridge. More specifically, it is thought that unlike on uniform PLL coating where the filopodia of a growth cone point in multiple directions, the ridge of the PLL line signals the filopodia to point in a single direction, which is the direction of the ridge. This convergence of growth directionality likely also explains the increased speed at which the extension occurs. Alternatively, it can be postulated that growth on peaks is advantageous because, in culture, the peak region is exposed to a different environment (e.g., broader exposure to oxygen and nutrients in the culture medium). Regardless of the mechanism involved, data provided herein support the functional use of raised surface features comprising or consisting of PLL to control the direction of growth of neurons on a solid substrate, without the need for “naked” regions to inhibit growth in other directions.

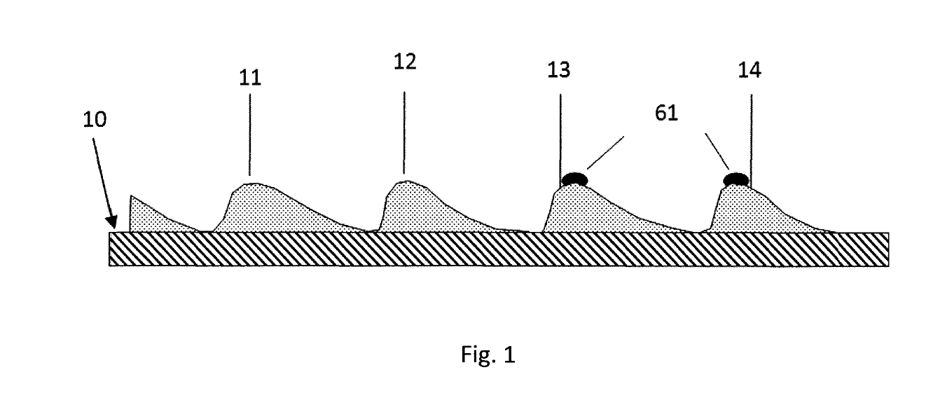

In preferred embodiments, coating of at least a portion of the solid substrate results from the process of differential deposition of PLL to form raised surface features. As such, the invention encompasses a one-step method for differential coating of a solid substrate to provide a solid substrate having one or more raised surface features comprising or consisting of PLL and having additional areas coated with PLL. More specifically, the one-step method results in deposition of PLL in raised surface features that are represented by wave-like cross-sections (see FIG. 1). The “peaks” of the features are raised above the surface of the solid substrate, and rise and fall in a definable manner. At portions of the surface away from the raised surface features, the surface is coated substantially uniformly by PLL. Where the surface has disposed on it two or more “peaks”, the valleys in between the “peaks” are coated with PLL as well, providing a surface that supports neuron attachment and growth.

The use of a single raised PLL surface feature thus allows for primary growth of a neuron along the length of the ridge. Growth along the ridge is not dependent on other surface features of the solid substrate (e.g., naked areas). In preferred embodiments, two or more raised PLL surface features are provided on a solid substrate to control the direction of growth of neurites. For example, in one basic configuration, a solid substrate having two parallel lines of PLL are disposed on the surface. One or more neurons are seeded on the solid surface and allowed to grow, and primary neurite growth proceeds along the ridge of the raised surface features. Neurite branches extending away from the axis of growth along the ridge of the PLL surface feature are substantially reduced, as compared to growth on uniformly coated surfaces, because the raised surface features provide a signalling mechanism for directionality of growth. The resulting neuron growth for all neurite branches is thus caused to be in the same direction, and is defined by the geometry of the PLL surface features.

The concept of multiple raised PLL surface features or lines is depicted in FIG. 1, which shows a sectional diagram of a solid substrate 10 having at least four raised PLL surface features (11, 12, 13, 14) resulting from differential deposition of PLL onto the surface of the solid substrate 10. Data from experiments performed on solid substrates created using differential deposition of PLL by differential evaporation to create multiple raised surface features revealed that the shape of the features is asymmetrical distributed, as illustrated in FIG. 1. Specifically, differential evaporation creates PLL lines having a cross-section that can be described as a wave-like pattern, which can explain why neurites grown on this surface strictly follow the PLL line. The neurite growth cone responds to the geometrical shape of the ridge of the PLL line as a directional signal. Depicted in FIG. 1 are neurites 61, which are shown as growing on the peaks of the raised surface features 11, 12, 13, and 14.

The distances between the deposited peaks can be any distance desirable, limited only by the ability to accurately deposit PLL in distinct lines. Typically, the distance between lines is on the order of micrometers (μm), such as from 0.5 μm to 10 μm, for example between 0.5 μm and 1.5 μm, or about 1 μm. However, it is to be noted that the effective width of a PLL line is smaller than the actual distance between lines because the effective width corresponds to the width of the peak of the wave (i.e., ridge). Using the methods of the present invention, peak widths on the order of the diameter of axons, dendrites, or growth cones can be created. PLL lines having peak widths of about 0.5 μm to about 1.5 μm can thus be disposed on solid substrates according to the invention. Currently known techniques for deposition of raised PLL surface features on solid substrates are incapable of achieving such short distances, being limited to distances between two paths or lines on the order of 40 μm.

In developing the method of controlling the growth of neurons on a solid substrate, it was surprisingly found that the method can control the rate of growth of neurons as well. More specifically, it has been discovered that growth of neurons along a PLL ridge results in an increase in the rate of extension of neurites, as compared to neurons grown on solid substrates having only uniform coating. While the precise mechanism underlying this discovery is not fully defined, it is possible that increased growth rate results from the focusing and convergence of the filopodia of a growth cone in the direction of the PLL ridge, leading to the unidirectional growth of all neurites of a neuron. That is, growth of neurites on uniformly coated solid substrates is directionless because the growth cone of the neurites has a plurality of filopodia pointing in multiple different angles, each searching for a signal for directional growth. The process of sending out multiple filopodia in search of a directional signal consumes energy and takes time, and results in random and slow growth on surfaces that do not contain directional signals (such as uniformly coated surfaces). In contrast, the raised PLL surface features of the present invention provide a clear signal and all filopodia point in the same direction, resulting in a much faster growth rate. Regardless of the molecular mechanism, the methods of the invention can provide for enhancement of growth rate of up to 20-fold or more (as compared to growth on uniformly coated solid substrates). As such, the invention encompasses methods for increasing the rate of growth of neurons on solid substrates, where the growth rate is increased from 2-fold to 20-fold, such as 5-fold, 10-fold, 15-fold, and 20-fold. Determination of growth rate can be by any suitable method. In exemplary embodiments, growth rate is determined by optical inspection of neurons, typically after staining or labeling with an appropriate substance, such as a fluorescent or luminescent compound.

A preferred method of the invention is a method for controlling both the directionality and rate of growth of neurons on a solid substrate. In general, the method comprises seeding neurons on a solid substrate having one or more raised surface features comprising or consisting of PLL that has been differentially deposited on the solid substrate, and maintaining the seeded solid substrate under conditions suitable for growth of the neurons for a sufficient amount of time for the seeded neurons to grow. Preferably, at least a portion of the solid substrate not differentially coated with PLL is uniformly coated with PLL. More preferably, the raised surface features comprise peaks having widths that are less than 10 μm. Typical conditions for growth of neurons in culture can be used, for example, growth in MEM culture medium plus supplemental elements in a humidified culture incubator at 37° C. with 5% CO₂. Typically, the solid substrate is also at least partially coated (e.g., fully and/or uniformly) with a substance that promotes neuron attachment, such as PLL. In exemplary embodiments, the solid substrate is at least 50% coated with a substance that promotes neuron attachment, such as PLL. According to the method, growth is any detectable increase in neurite length (after initial attachment and settling of cells on the substrate). According to this embodiment of the method of the invention, rapid and controlled growth of neurons is achieved, providing neurons highly suited for in vitro study and in vivo therapeutic use.

The presently disclosed technology has applicability in the medical arts. Specifically, the present invention provides for methods and products for medical treatment of patients suffering from neural damage. In embodiments, the invention relates to use of products of the invention for medical treatment of patients. As such, the invention provides a method of treating a subject in need of neuron repair or replacement, where the method includes implanting a medical device into the subject at a site of neuron damage or loss. According to the method, the medical device includes a solid substrate having disposed on a surface one or more raised surface features comprising PLL in a pre-defined geometric shape, as discussed above. Further, implanting of the device allows one or more neurons at the site of neuron damage or loss to physiologically interact, either directly or indirectly through another neural cell, with a natural target cell for the neuron. The method can be thought of as resulting in reformation of a severed neural connection. In some embodiments, implanting of the device allows one or more neurons at the site of neuron damage or loss to grow on and over the surface of the solid substrate and physiologically interact with a natural target. This is accomplished as a result of the neuron growth-controlling characteristic of solid substrates according to the invention. Although the method can be practiced by implanting a device simply having the physical characteristics mentioned above, in embodiments, the device is provided in a form comprising one or more neurons that were grown on the solid substrate in vitro prior to implantation of the device in the subject. To enhance the regenerative effect of the treatment methods, in some embodiments, the medical device includes a solid substrate that has one or more raised surface features comprising PLL in a pre-defined geometric shapes disposed on two surfaces. For example, a solid substrate can be fabricated of a biodegradable, flexible material, such as a biodegradable poly glycolic acid (PGA) film differentially coated with PLL. Medical use of PGA film is described in U.S. Pat. No. 5,853,639, for example. Techniques for patterning PGA film are known in the art. After coating, and optional neuron growth, the PGA film can be rolled into a tube to form a “nerve bundle” for nerve repair, where neurons can grow on the internal surface, the external surface, or both.

It is known in the art that engineered axon bundles can be successfully used for neuron repair. (See, for example, Tissue Engineering: Part A. Vol 15, Number 7, 2009). To produce the axon bundle for nerve repair, these researchers used a sophisticated computerized device to stretch axons for days during tissue culture. Although the reported work is complex, the work demonstrates the feasibility of implanting nerve bundles to repair severed nerves. The present method of generating neuron bundles is significantly simpler to implement and the resulting product more amenable to commercial manufacturing. Its characteristics make it highly suitable for use in vivo for treatment of a patient in need.

The methods of the invention use solid substrates having differentially deposited PLL surface features. As such, the present invention includes a wide range of products based on such solid substrates. In a basic form, a product of the invention is a solid substrate having a surface on which one or more raised surface features comprising or consisting of PLL are present. In preferred embodiments, the surface also includes at least partial coating with PLL at regions of the solid substrate that are not differentially coated with PLL. Coating at regions other than where PLL is differentially deposited can be a uniform and/or full covering of PLL. As discussed above, the solid surface is not particularly restricted, and can include any suitable natural or man-made substance or combination of substances. The products can be used both in vitro and in vivo. Uses in vitro typically relate to research on neuron physiology, growth, and function. Uses in vivo typically relate to therapeutic treatments. As such, products for in vitro applications generally include a solid substrate fabricated from plastic or glass, such as currently known in the art for use in growth and study of neurons. Products for in vivo applications generally include a solid substrate that is biologically tolerable and acceptable for short-term or long-term implantation into a body. Numerous biocompatible substances are known in the art, and the practitioner is free to select a particular solid substrate based on relevant parameters for each particular application of the technology.

Among the many forms products of the invention may take, mention can be made of the following non-limiting examples: microscope slides; coverslips; microtiter plates or wells; culture dishes or plates; impermeable, semi-permeable, or permeable polymeric membranes (e.g., nylon), microarrays, microfluidic channels, sheets, tubes, threads, beads, needles or cannula, and stents.

The size and shape of the products are not particularly limited, and the practitioner is free to select an appropriate size and shape for a particular application. Most applications will not require a product having a length greater than about 0.5 cm. However, because neurons are capable of growing to a relatively long length, the size of the product can be on the order of 10 cm or longer, such as 50 cm, or 100 cm.

While the products of the invention can be provided simply in the form of a solid substrate having surface features, in some embodiments, the product includes one or more neurons attached to a surface of the solid substrate. In some cases, one or more neurons are attached to two surfaces of the solid substrate, such as to an upper and a lower surface. For medical applications, the product can be a device for implantation into a patient, where the device can include a solid substrate on which multiple neurons are attached and growing.

Various techniques for manufacturing products according to the invention may be used. Among the techniques, mention may be made of use of differential evaporation. More specifically, the method includes differentially depositing PLL on one or more surfaces of the solid substrate to form raised surface features on the surface(s). The process of differential evaporation includes immersing a solid substrate into a PLL solution to completely cover the area on the solid substrate desired, and allowing the molecular events that occur during evaporation to form functional raised surface features on the solid substrate. In essence, the portion of the solid substrate to be treated is completely exposed to PLL, and molecular movement of PLL at the liquid-air interface generates the raised surface features. The process generates products having advantageous properties, including, but not limited to, raised surface features having widths of less than 10 μm, parallel lines of raised surface features disposed within 10 μm or less of each other, and raised surface features having relatively smooth surfaces, as compared to those created using micro-printing or micro-patterning techniques. In the differential evaporation process according to the present invention, the natural movement and interaction of substances at the molecular level is used to form raised surface features. This mechanism is in contrast with the physical transfer of PLL from a stamp or printing head to the surface of a solid substrate, which is used in micro-printing and micro-patterning techniques known in the art.

The process of differential evaporation can be thought of as a one-step process for depositing PLL in raised surface features and coating other portions of the surface of the solid substrate uniformly or substantially uniformly with PLL. In embodiments, more than one surface (e.g., upper surface and lower surface; inner surface and outer surface) of the solid substrate is coated differentially and/or uniformly. Where the products include neurons, standard techniques for seeding neurons onto solid surfaces may be used.

The method of making products includes differentially depositing PLL on a solid substrate in a pre-defined geometrical pattern or shape. The pattern of PLL surface features defines the growth path of neurons on the product. For straight geometrical lines, differential evaporation can be used directly to form lines on the surface of the solid substrate. Alternatively, special devices, such as micro-beads or micro-rods, can be used to interfere with the movement of PLL on the substrate so that circular or U-shape PLL lines can be formed on the substrate, respectively.

The invention thus encompasses use of differential PLL deposition on a solid substrate to create products and to control growth pattern and/or rate of growth of neurons. It likewise encompasses use of differential PLL deposition on a solid substrate to create products for use in research and medicine. For example, the invention encompasses use of a product of the invention to perform research on neuron growth and function. Additionally, the invention encompasses use of a product to treat neural injury or neural deficiencies, abnormalities, or degradation. Use of the products as research tools is likewise encompassed by the invention. Such research tools can take the form of neuron culture products (e.g., plates, slides, cell culture chambers, etc.), and can be coated with PLL in defined patterns to direct neurite growth. The geometry of these patterns can determine the length, direction, and growth rates of neurites. The patterns can also be custom-made to tailor to a researcher's specific experimental requirements. Various proteins or other bioactive substances of interest can also be embedded into the substrate to study their effect on neurite properties.

One exemplary use of the methods and products of the invention is in the area of drug discovery. That is, micro-patterned neuron chips can be designed for drug screening. Such chips can be used to run High Throughput Screening (HTS) assays. Drugs screened can be in solution or in the substrate, although screening assays that focus on exogenously supplied substances for assay are less expensive and more versatile. Non-limiting examples of uses of the technology are detailed below.

The present invention represents the first demonstration of the neurite guidance properties of patterned PLL. The invention enables the fabrication of printed chips of micropatterned PLL for directed axon and dendrite growth, which can be used, for example, in research into guidance mechanisms or for drug screening of neuroactive compounds.

### EXAMPLES

The invention will now be further explained by the following Examples, which are intended to be purely exemplary of the invention, and should not be considered as limiting the invention in any way.

### Example 1

### Method of making PLL Lines by Differential Evaporation

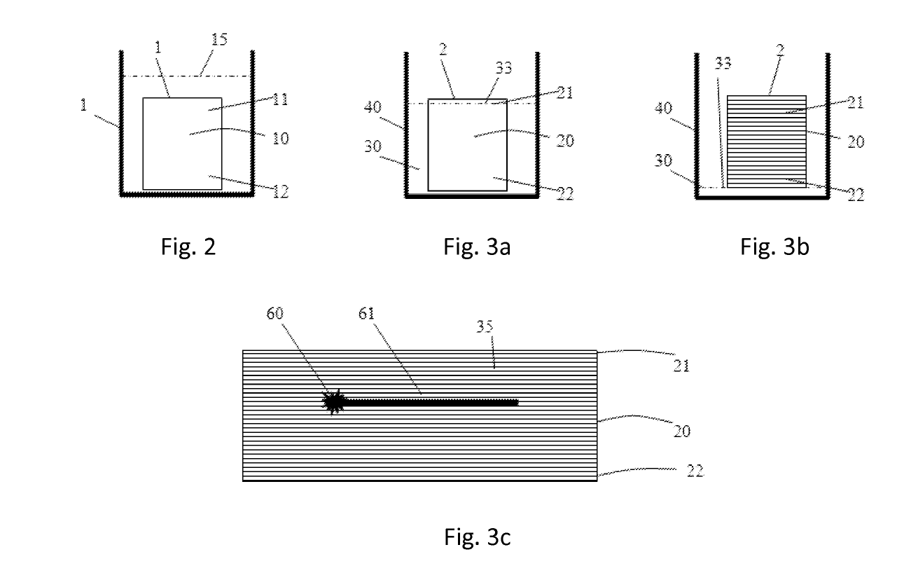

Differential evaporation is a novel method for coating a surface with PLL to create linear tracks, which reveals the ability of PLL to control the directional growth and branching of neurites. All prior uses of PLL coating of solid substrates involves uniform coating. Such a process is depicted in FIG. 2. In that process, a cover glass 1 has a surface 10 between a top 11 and a bottom 12. At a starting time point, surface 10, from top 11 to bottom 12, is immersed completely in a solution of PLL 15. At an ending time point, cover glass 1 is quickly removed from PLL solution 15. The resulting cover glass is uniformly coated on surface 10 between top 11 and bottom 12, showing no coating variation.

Although embodiments of the present invention include coating of at least a portion of a solid substrate prior to differential deposition of PLL on the solid substrate surface, the present invention preferably includes differential deposition of PLL on the solid substrate as a means for producing one or more raised PLL surface features and coating of at least a portion of the remaining surface with PLL, all in a one-step process. Differential deposition of PLL and coincidental uniform coating with PLL in a one-step process by way of differential evaporation is shown in FIGS. 3a and 3b. More specifically, FIGS. 3a and 3b illustrate the starting time point (FIG. 3a) and ending time point (FIG. 3b) of PLL coating via differential evaporation. A solid substrate 2 has at least one surface 20 between a first point 21 and a second point 22. To start coating surface 20, solid substrate 2 is placed vertically in a container 40. A PLL solution 30 is added to container 40 to contact surface 20. A level 33 of PLL solution 30 is adjusted to a height adjacent to first point 21. PLL solution 30 is allowed to stay in contact with surface 20 for an amount of time adequate to cause a raised surface feature to be formed by way of evaporation at the air-solution boundary (i.e., level 33). At a given time point, level 33 of PLL solution 30 is adjusted from first point 21 to a lower point, and deposition of PLL on surface 20 is allowed to proceed as at first point 21. This process of evaporative deposition and adjustment of level 33 is repeated multiple times to provide a solid substrate 2 having multiple raised surface features of PLL from first point 21 to second point 22, as shown in FIG. 3b. When the final raised surface feature is completed, solid substrate 2 is removed from PLL solution 30. Uniform coating of the remainder of solid substrate 2 occurs as per standard uniform coating (e.g., FIG. 2). The principles of formation of the coated solid substrate are applicable to any shape or size of solid substrate, and the process can be performed on multiple solid substrates per reaction container.

The evaporative deposition of PLL described above and depicted in FIGS. 3a and 3b uses physical and chemical properties of PLL in solution and the phenomenon of evaporation to create peaks or ridges of PLL on the surface of a solid substrate. More specifically, an important feature of water is its dipolar nature, which generates cohesive forces that hold water molecules together and adhesive forces that draw and hold water molecules to hydrophilic surfaces. Water that is in contact with other substances has a surface tension at the site of contact, which results from the cohesive forces of the water. In the process of creating differentially coated solid substrates of the invention, PLL is in contact with the surface of the solid substrate. At the liquid-air barrier, PLL is deposited as a result of evaporation of the water, whereas beneath the water surface, PLL remains in solution. Deposition at the water-air barrier is allowed to continue for a given amount of time, at which point the level of PLL solution is lowered (either by lowering the solution level or raising the solid substrate) and a second line of PLL deposition is started. This process can be repeated multiple times to create multiple raised surface features. Using this differential evaporation technique, parallel lines of PLL having a spacing of less than 1.5 μm have been created.

The differentially coated solid substrate can be used for controlled growth of neurons along the lines. This concept is depicted in FIG. 3c, in which a neuron is depicted as growing along the raised surface features. According to the figure, during culture, a neuron 60 re-generates a neurite 61. The growth direction of neurite 61 is controlled by line 35. Neurite 61 remains in a single and long morphology because line 35 exerts a potent force to guide its growth.

### Example 2

### Isolation and Seeding of Neurons, and Method of Controlling Direction of Neuron Growth

Tissues were removed from brains of mouse pups at one day of age. Neurons in a piece of brain tissue were first separated via surgical isolation. Then, individual neurons were dispersed from that piece of brain tissue via proteinase digestion. All axons of the neurons were destroyed during the process. The damaged neurons were then plated on PLL-coated glass coverslips of two types. The first type of coverslip was uniformly coated with PLL. The second type of coverslip was one according to the present invention, in which PLL was differentially deposited in curved lines. The coverslips with attached neurons were immersed in culture medium and placed in a CO₂ incubator for axon regeneration. After 7 days, regenerated axons were fixed with paraformaldehyde and visualized by immunochemistry labeling.

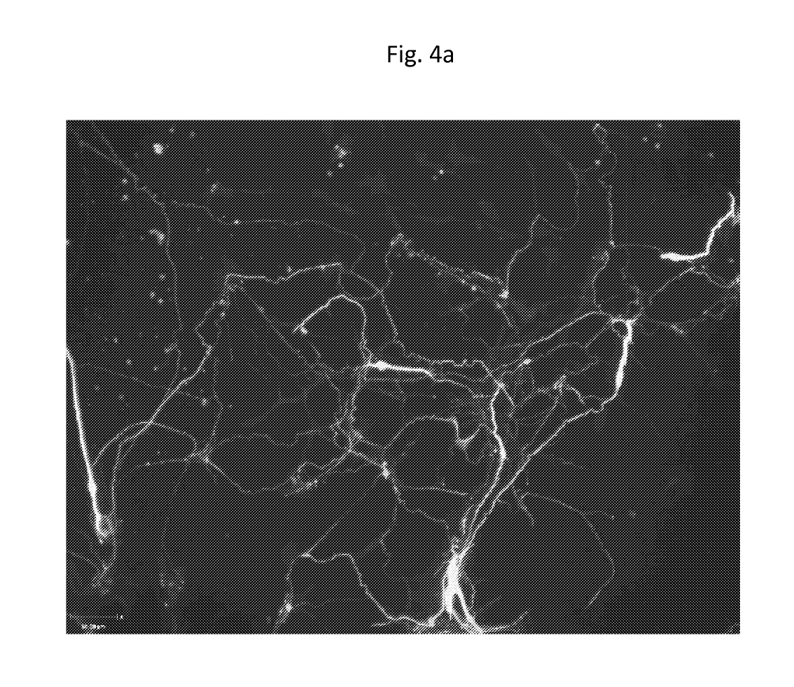

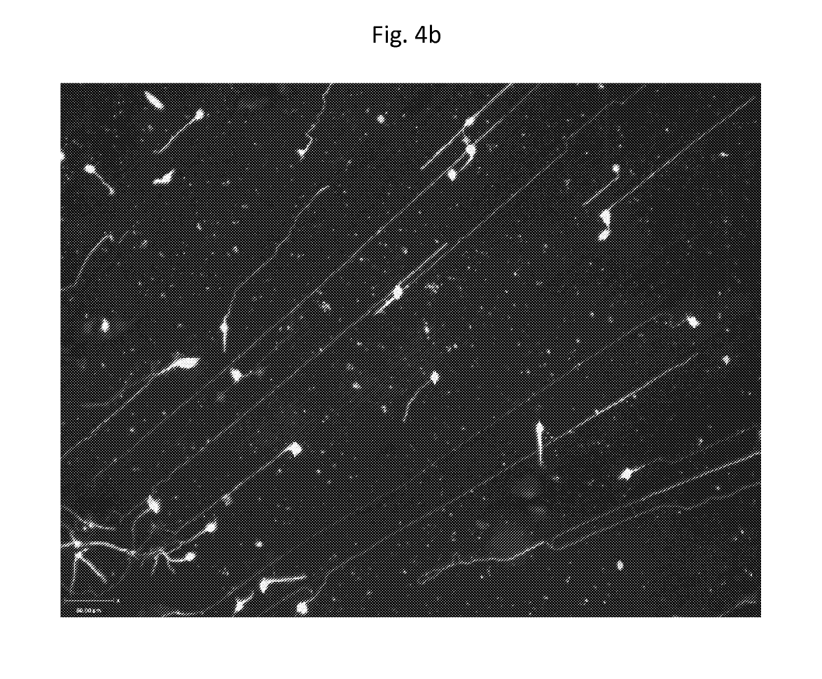

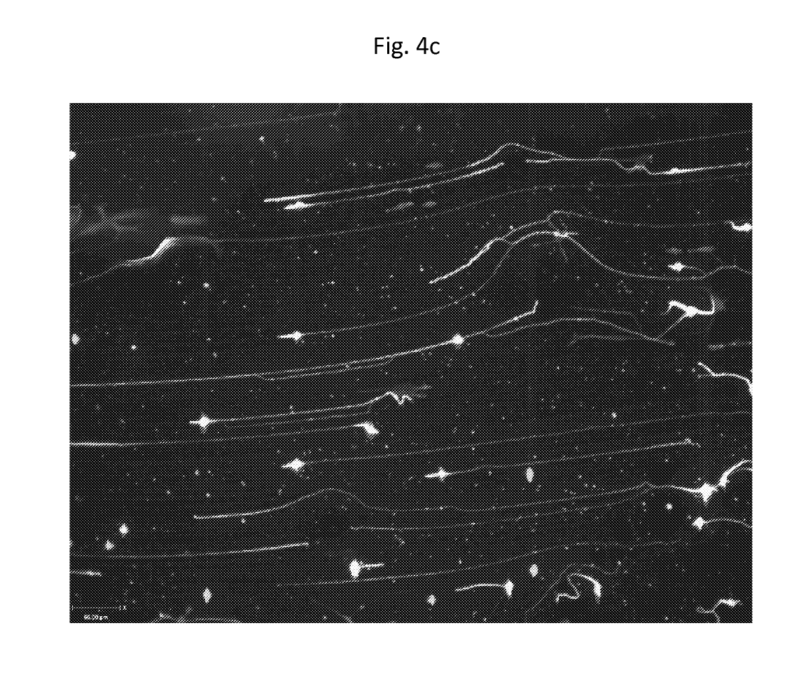

As shown in FIG. 4a, when PLL was uniformly coated on glass according to traditional protocols, the regenerated axons did not display directed growth patterns and instead showed a random pattern of growth with multiple branches around the body of the neuron. However, when PLL was coated on the coverslip to form geometrically oriented lines, the regenerated axons grew along the direction of the lines of PLL and showed little or no axon branching (see FIGS. 4b and 4c).

### Example 3

### Increased Rate of Growth of Neurons

In analyzing the results obtained in Example 2, it was observed that neurites of neurons grown on the solid substrates of the present invention were significantly longer than those of neurons grown on uniformly coated substrates. To investigate this observation further, neurons were again seeded onto uniformly coated substrates and substrates coated according to the present invention, and the rate at which the neurons grew was monitored. It was determined that neurons grown on solid substrates according to the present invention showed a growth rate that was about ten times greater than neurons grown on uniformly coated substrates.

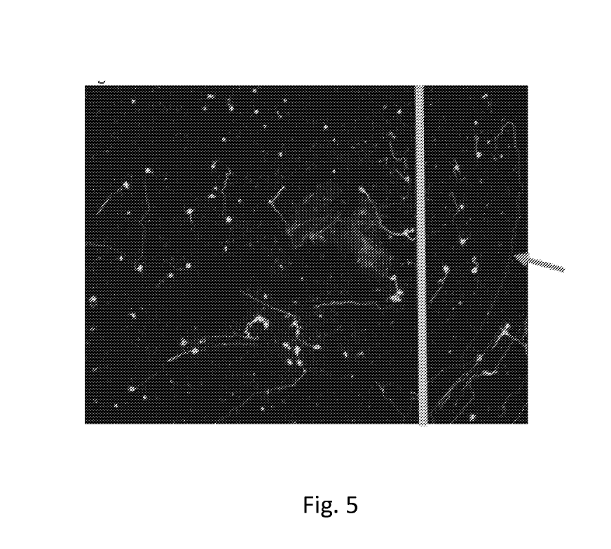

More specifically, neurons were obtained and seeded according to the procedure in Example 2, including the following details. A coverslip was coated in two ways, half by uniform PLL coating and half by differential PLL lines. Neurons seeded on the single coverslip were cultured in a single well using a single culture medium. After two days of growth, the neurons were observed for size, numbers, and direction of growth. The results are shown in FIG. 5. In the 2-day culture, the neurons located on the uniform PLL coated areas on the left of the figure show short neurite lengths. In contrast, the cells grown on the PLL pattern on the right of the figure show increased neurite growth. There is an approximate 10-fold increase of neurite length when compared with the uniform coated control. It is postulated that the electrochemical properties of the PLL line signals the neurite growth cone to develop in a specific direction. In contrast, the control area of uniform PLL coating does not provide such signaling and the growth cone alternates in direction, resulting in a net gain of neurite growth that is shorter and growth rate that is slower.

An important parameter to measure when studying neural circuits is neurite growth rate. Using standard culturing methods, where neurites branch out irregularly, it is challenging to measure neurite length and therefore growth rate. The present invention enables neurites to grow in a defined direction, for example linearly, such that neurite growth can be easily measured and neurite-neurite interactions can be studied with specificity. As such, neurite growth rate measurements can easily be automated. Furthermore, when cultured linearly, the neurites grow up to or greater than ten times faster than under standard culturing conditions. Currently, measuring neurite growth rates takes approximately ten days to culture cells, immunohistochemically stain them, image them via microscope, and analyze the images using computer software. The method of the present invention can reduce the measurement time to 48 hours and be completely automated for use in high throughput assays for basic research or drug screening.

### Example 4

### High Throughput Screening Assays and Platform

One exemplary embodiment of the invention is a platform for high throughput screening (HTS) assays of neuronal growth. A platform for this type of use is desired in both the scientific and the pharmaceutical fields. However, to date, there is no commercial product available to achieve such a platform. Attempts have been made to create microchips for neuron assays using bioactive proteins. Laminin, for example, was used to form a micro-pattern to direct growth of axons in a grid pattern. However, the use of laminin protein as a patterning agent remains restricted within the basic research arena, without commercial development, likely due to the fact that laminin, a protein found in extracellular matrix of animals, is a complex molecule with a molecular weight of 900 kD and made up of three separate parts, called A, B1, and B2 chains. This complexity argues against its use. Furthermore, human laminin costs $2300-$8000 for a single milligram. In addition, laminin is a biological active material with a limited shelf life requiring storage at −80° C. Further, its bioactivity is lost with changes in 3-D structure.

In comparison to laminin, chemically synthesized PLL, as utilized in this invention, costs only $7 per milligram, which is 0.136% of the average cost of laminin in the same quantity, providing a cost reduction of over 700-fold. In addition, PLL is stable at room temperature and has a shelf life of 1 year or more. These features of PLL establish tremendous commercial advantages in terms of both feasibility and profitability over the use of laminin protein.

The present Example discusses HTS platforms and assays. Numerous configurations are feasible, and numerous different properties of neurons can be taken advantage of to identify substances that have biological effects on neurons. Various advantageous properties of seeded solid substrates of the present invention, discussed herein, can be used to provide HTS platforms and assays.

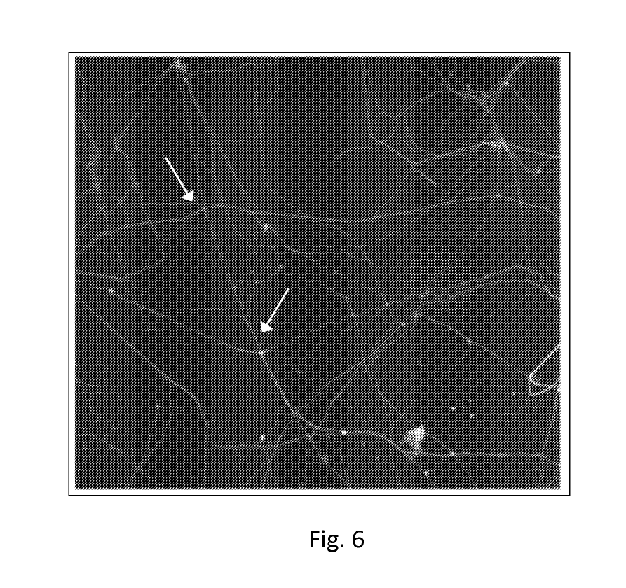

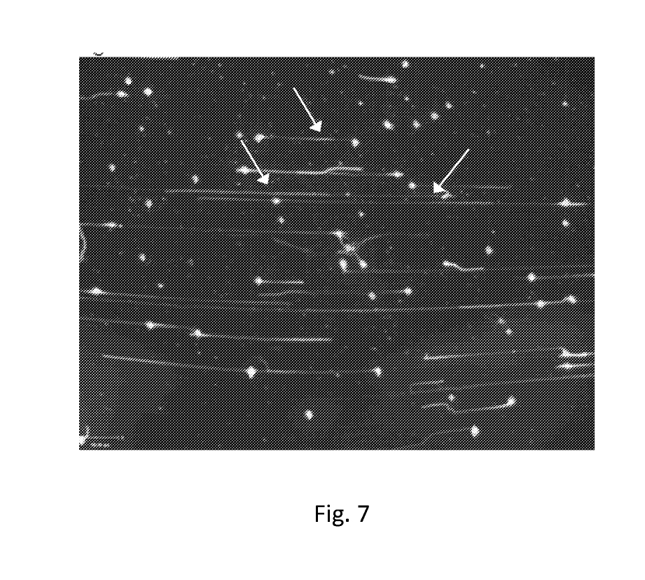

Initially, it was important to confirm that neurons grown on substrates of the invention could successfully interact in a manner that is suitable for an HTS assay. As such, experiments were performed to confirm that detection of neurotransmitter activity would be possible. The results are depicted in FIGS. 6-9. Specifically, a comparison between neurons grown according to a standard protocol and neurons grown according to the present invention was made to determine if the present invention provided an improvement in the number and type of neuronal interactions. FIG. 6 shows a photograph of neurons grown under standard conditions (i.e., on a solid substrate that is uniformly coated with PLL). As can be seen from the figure, growth direction is random and neurites intersect each other at various points (referred to herein as “point contacts”), which are also randomly distributed. In contrast, as shown in FIG. 7, growth of neurons according to the present invention show neurites in the same line, which enter into contact along their length, creating a defined line of contacts (referred to herein as “line contacts”). The fact that line contacts are created in a defined geometry (in this case a straight line), allows for a uniform, reproducible HTS platform to be created in many different configurations.

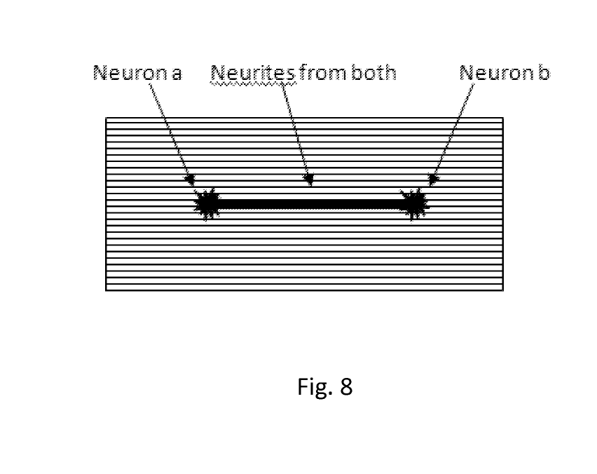

The results depicted photographically in FIG. 7 are interpreted schematically in FIG. 8. FIG. 8 shows the growth of two neurons (“neuron a” and “neuron b”) in a single line on the surface of the solid substrate. Neurites from each neuron interact along the line (depicted by stars in FIG. 8 and as balls in FIG. 7). In view of this controlled interaction along a pre-defined geometry (in this case a line), assays and platforms can be created that identify the effects on one neuron of treatment of the other neuron. Likewise, the effect on both neurons of treatment of one or both neurons can be assayed. For example, an HTS neuron chip can be readily designed with a stimulating electrode contacting “neuron a” and a recording electrode contacting “neuron b”. The length contact between neurons a and b along a PLL line provides numerous synapses, which will generate a strong signal of neurotransmitters in response to stimuli, such as test compounds (e.g., drugs or other bioactive compounds that affect neuron growth, death, and/or function), thereby amplifying the measurable signal between the two neurons and the sensitivity of the test. As mentioned above, the non-random and reproducible nature of the growth and interaction of neurons can be used as a powerful tool in developing assays for investigating any number of effects of stimuli on neurons.

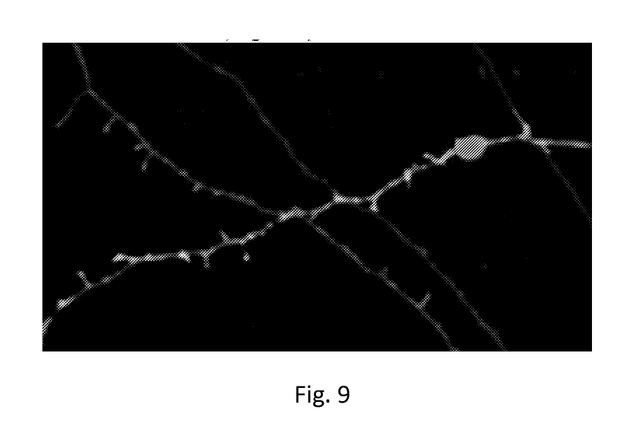

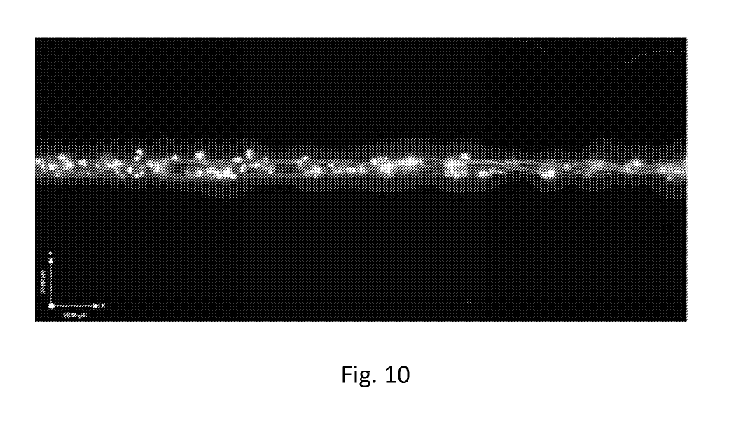

Having established that line contacts could be created, experiments were then performed to determine if multiple line contacts could be created per PLL line between multiple neurons. It was expected that multiple point contacts, resulting from growth of neurons on standard conditions, would be minimal and randomly distributed. Conversely, neurons grown according to the present invention were expected to provide controlled, defined connections within a single line. Such a situation has clear advantages in improving signal strength, reproducibility, and reliability. The results of experiments comparing point contacts to line contacts are shown in FIGS. 9 and 10. FIG. 9 shows point contacts created by random growth of neurons under standard conditions. Very few point contacts between four neurites can be seen when cells were stained using a marker for GABA-ergic neuronal terminals. In contrast, FIG. 10 shows that numerous line contacts (shown as spherical structures) are detected between multiple neurites grown in close apposition along a single PLL line. Like the other features discussed herein relating to growth of neurons, the numerous line contacts resulting from geometrically defined and controlled growth provide an advantage in HTS assay platforms by providing strong, reproducible, reliable, and controllable assay readouts.

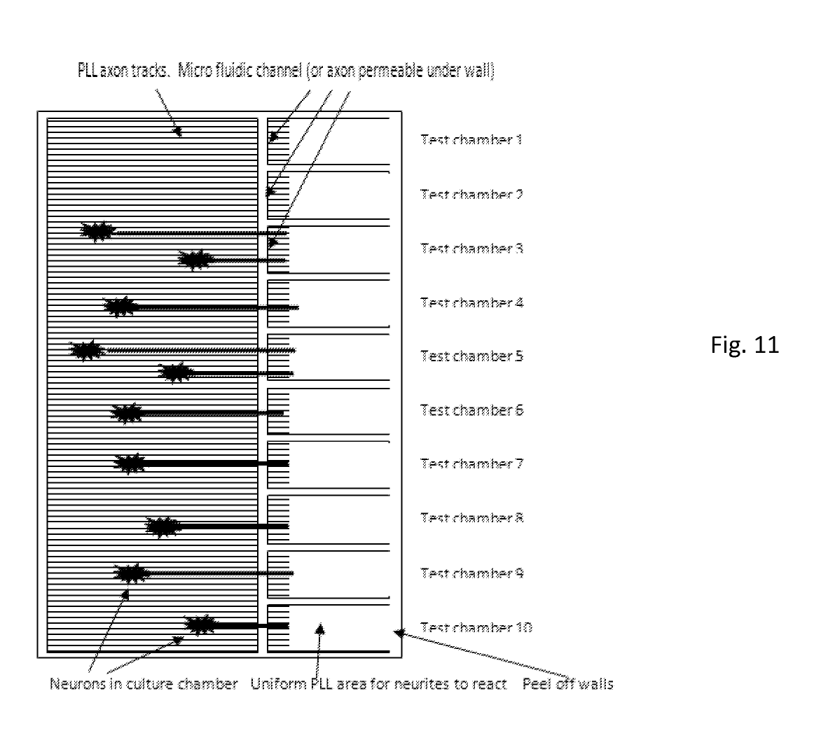

An exemplary HTS assay platform is provided as FIG. 11. As can be seen in the figure, the platform is divided into two main sections, one for controlled neuron growth along lines and the other for neuron interaction with test compounds. According to this exemplary embodiment, primary neuron growth is along PLL lines according to the invention. Neurites terminate at one end at an area uniformly coated with PLL, which allows for neurite growth (not directionally controlled) and interaction with test substances. Although only one neuron per test chamber is depicted, it is to be understood that, in practice, all or substantially all of the lines are populated with neurons, and that each line is populated with multiple neurons.

In practice, neurons are seeded onto the culture chamber portion of the platform and permitted to grow such that neurites extend along the lines and enter the reaction or test chambers. Upon achieving suitable growth into the test chambers, test substances are introduced into the test chambers and the effect of the substances on the neurons is determined. Although numerous ways of determining the effects on the neurons are possible, often detection will be by way of detection of release of certain chemicals or by way of changes in electrical conductivity.

Like other HTS platforms and assays, the present HTS platforms and assays are widely variable and can be adapted to any number of inquiries. For example, in the assay platform specifically depicted in FIG. 11, the platform is configured with one culture chamber and test chambers. Within this context, the platform can be used to test 10 different drug candidates (one per chamber), to test one drug candidate at ten different concentrations, to expose neurites to one drug for ten different time periods, or to test one drug alone and with nine booster doses. Of course, as with other HTS platforms, the neurites in each test chamber can be exposed to a complex mixture of substances to determine if the mixture contains one or more biologically active substances. Where a mixture is determined to include one or more active substances, the substances in the mixture can be separated (partially or completely) and re-assayed to identify the particular substance having activity.

Fabrication and use of HTS platforms according to the present invention provides numerous advantages and addresses many needs in the art, which could not be addressed using currently available technology. The following are some of the advantages that can be recognized. First, each measurement taken can be obtained much faster than if standard culturing conditions were used because PLL differential deposition has now been shown to increase neurite growth rates ten-fold or so. Increased growth rate reduces the time required to achieve growth from the culture chamber to the test chamber, and thus lowers the time and cost of preparing the platform for use. Furthermore, a very high number of measurements can be obtained per chip given that all or substantially all neurons on the chip reach the testing chambers, or are connected to neurons that have reached the test chamber. In addition, there is a very low probability of error due to limited variability (i.e., there is high data confidence) between neurite growth conditions: all neurons are in one culture condition and neurites grow uniformly without branching into the testing chamber where they are exposed to drug candidates, in solution or in the substrate. Likewise, neurite behavior can be easily imaged and measured, and the output can be further analyzed using computer software. The increased sensitivity and efficiency conferred by this invention, results in a significant reduction of media, test compound and staining reagents needed to obtain the measurements described above.

### Example 5

### Medical Devices

When nerves are cut or interrupted due to spinal cord injury or amputation, for example, the damaged neurons retain the potential to regenerate their neurites. However, most of the regenerating neurites fail to reach to their targets due to the interference of scar tissue and the lack of guidance to properly navigate towards their target cells, resulting in functional disability. The present technology can be applied for in vivo use, to guide severed neurites toward target cells. For example, medical devices, such as stents, biocompatible films, tubing, or other implantable scaffolds can be differentially coated with PLL as described above. The coated implant then can be implanted directly in the body to connect each severed extremity of a nerve through in vivo neurite extension along PLL lines of the device. Alternatively, neurons can be grown in vitro on the implant prior to engraftment. The rapid neurite growth allows for quick reconnection of nerves and their targets in vivo and rapid generation of a “nerve bundle” in vitro for use in nerve repair surgery. Another medical application uses the inhibitory property of PLL of neurite branching, to restrict unwanted nerve growth by placing a fully coated implant to “cap” severed nerve ends. This can address symptoms such as phantom pain.

Like the HTS assay platforms, medical devices of the invention have advantages that address needs in the art. For example, they are relatively inexpensive to make, they have relatively long shelf-lives at room temperature (when not seeded with neurons), they are easy to store and handle, and they are easily produced through high-volume chemical synthesis methods.

It will be apparent to those skilled in the art that various modifications and variations can be made in the practice of the present invention and in construction of devices according to the invention without departing from the scope or spirit of the invention. Other embodiments of the invention will be apparent to those skilled in the art from consideration of the specification and practice of the invention. It is intended that the specification and examples be considered as exemplary only.

1. A solid substrate having disposed on a surface one or more raised surface features comprising poly D/L lysine (PLL) in a pre-defined geometric shape, wherein the raised surface features have a wave-like cross-section.

2. The solid substrate of claim 1, wherein the widths of the peaks of the raised surface features are between about 0.5 μm and about 1.5 μm.

3. The solid substrate of claim 1, having two or more raised surface features comprising PLL.

4. The solid substrate of claim 1, wherein the geometric shape is a straight line or a curve.

5. The solid substrate of claim 1, wherein two surfaces of the solid substrate have differentially deposited raised surface features comprising PLL.

6. A high-throughput assay platform comprising the solid substrate of claim 1.

7. A medical device comprising the solid substrate of claim 1.

8. The solid substrate of claim 1, wherein the PLL covers more than 50% of the area of the surface treated with PLL.

9.-13. (canceled)

14. A method for controlling the direction of growth of neurons, said method comprising:
growing one or more neurons on a solid substrate having disposed on a surface one or more raised surface features comprising PLL in a pre-defined geometric shape,
wherein the raised surface features have a wave-like cross-section, and
wherein the raised surface features control the direction of growth of the neurons.

15. The method of claim 14, wherein the method comprises:
seeding one or more neurons on the solid substrate, and
maintaining the seeded solid substrate under conditions suitable for growth of neurons.

16. The method of claim 14, which is a method of increasing the rate of growth of neurons as compared to neurons grown on other solid substrates.

17. The method of claim 14, wherein the widths of the peaks of the raised surface features are between about 0.5 μm and about 1.5 μm.

18. A high-throughput screening (HTS) assay for a substance that affects neuron activity, said method comprising:
exposing a neuron growing on an HTS platform to a test substance,
wherein the HTS platform comprises a solid substrate having a culture chamber and a test chamber, the culture chamber having a surface on which is disposed one or more raised surface features comprising PLL in a pre-defined geometric shape for controlled directional growth of the neuron into the test chamber, wherein the raised surface features have a wave-like cross-section, and
determining if the test substance causes a change in the activity of the neuron.

19. The method of claim 18, wherein the change in activity of the neuron is release of a chemical.

20. The method of claim 18, wherein the culture chamber has two or more parallel raised surface features comprising PLL.

21. The method of claim 18, wherein the widths of the peaks of the raised surface features are between about 0.5 μm and about 1.5 μm.

22.-26. (canceled)

27. A one-step method for making a solid substrate for controlled growth of neurons, said method comprising:
immersing at least a portion of the solid substrate in a liquid composition comprising PLL to define a first liquid-air interface on a surface of the solid substrate; and
maintaining the solid substrate in the liquid composition for an amount of time sufficient to cause deposition of PLL onto the surface of the solid substrate at the first liquid-air interface,
wherein the deposited PLL at the first liquid-air interface forms a raised surface feature on the solid substrate that has a wave-like cross-section.

28. The method of claim 27, further comprising:
adjusting the position of the solid substrate in the liquid composition to define a second liquid-air interface on the surface of the solid substrate; and
maintaining the solid substrate in the liquid composition for an amount of time sufficient to cause deposition of PLL onto the surface of the solid substrate at the second liquid-air interface,
wherein the deposited PLL at the second liquid-air interface has a wave-like cross-section.

29. The method of claim 28, further comprising:
repeating the adjusting and maintaining steps one or more times to create multiple raised surface features having wave-like cross-sections.

30. The method of claim 27, wherein the width of the peak of the raised surface feature(s) is between about 0.5 μm and about 1.5 μm.
